SEQUENCE LISTING (26 sequences)

SEQ ID NO: 1 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 2 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 3 - protein, 31 aa, Artificial Sequence
  epitope 1 related to Clostridium difficile toxins A and B
  VARIANT (14)..(14)
  VARIANT (18)..(18): Xaa can be any naturally occurring amino acid moiety or is a direct bond between the adjacent amino acid moieties
  VARIANT (22)..(22): Xaa is an amino acid moiety selected from the group consisting of Ser, Thr, Tyr, Cys, Asn, Gln, Lys, Arg, His, a selenocysteinyl moiety, a pyrrolysinyl moiety, Asp and Glu
  VARIANT (29)..(29): Xaa is an amino acid moiety selected from the group consisting Ala, Gly and Ser
  VARIANT (31)..(31): Xaa is an amino acid moiety selected from the group consisting of Gly, Ala, Val, Pro, Leu, Ile, Met, Trp and Phe
ANQYEVRINSEGRXELLXHSGXWINKEEXIX

SEQ ID NO: 4 - protein, 10 aa, Artificial Sequence
  epitope 2 related to Clostridium difficile toxins A and B
  VARIANT (4)..(4): Xaa is an amino acid moiety selected from the group consisting of Gly, Ala, Val, Pro, Leu, Ile, Met, Trp and Phe
  VARIANT (5)..(5): Xaa is an amino acid moiety selected from the group consisting of Tyr, Gly, Ala, Val, Pro, Leu, Ile, Met, Trp and Phe,
GESXXVETEK

SEQ ID NO: 5 - protein, 18 aa, Artificial Sequence
  epitope 3 related to Clostridium difficile toxins A and B
  VARIANT (1)..(1): Xaa is an amino acid moiety selected from the group consisting Ala, Gly and Ser
  VARIANT (2)..(2): Xaa is an amino acid moiety selected from the group consisting of Gly, Ala, Val, Pro, Leu, Ile, Met, Trp and Phe
  VARIANT (6)..(6): Xaa is an amino acid moiety selected from the group consisting of Gly, Ala, Val, Pro, Leu, Ile, Met, Trp and Phe
XXKVQXYAQLFSTGLNTI

SEQ ID NO: 6 - protein, 21 aa, Artificial Sequence
  epitope 4 related to Clostridium difficile toxins A and B
  VARIANT (2)..(2): Xaa is an amino acid moiety selected from the group consisting of Gly, Ala, Val, Pro, Leu, Ile, Met, Trp and Phe
  VARIANT (4)..(4): Xaa is an amino acid moiety selected from the group consisting of Gly, Ala, Val, Pro, Leu, Ile, Met, Trp and Phe
  VARIANT (20)..(20): Xaa is an amino acid moiety selected from the group consisting of Gly, Ala, Val, Pro, Leu, Ile, Met, Trp and Phe
LXPXAGISAGIPSLVNNELXL

SEQ ID NO: 7 - protein, 15 aa, Artificial Sequence
  epitope 5 related to Clostridium difficile toxins A and B
DDLVISEIDFNNNSI

SEQ ID NO: 8 - protein, 23 aa, Artificial Sequence
  epitope 6 related to Clostridium difficile toxins A and B
  VARIANT (11)..(11): Xaa can be any naturally occurring amino acid moiety or is a direct bond between the adjacent amino acid moieties
  VARIANT (12)..(12): Xaa is an amino acid moiety selected from the group consisting of Ser, Thr, Tyr, Cys, Asn, Gln, Lys, Arg, His, a selenocysteinyl moiety, a pyrrolysinyl moiety, Asp and Glu
  VARIANT (19)..(19): Xaa is an amino acid moiety selected from the group consisting Ala, Gly and Ser
  VARIANT (23)..(23): Xaa is an amino acid moiety selected from the group consisting of Ser, Thr, Tyr, Cys, Asn, a selenocysteinyl moiety, a pyrrolysinyl moiety and Gln
MEGGSGHTVTXXIDHFFSXPSIX

SEQ ID NO: 9 - protein, 15 aa, Artificial Sequence
  epitope 7 related to Clostridium difficile toxins A and B
  VARIANT (12)..(12): Xaa is an amino acid moiety selected from the group consisting of Lys, Arg and His
PGLRSLENDGTXLLD

SEQ ID NO: 10 - protein, 59 aa, Artificial Sequence
  epitope 8 related to Clostridium difficile toxins A and B
  VARIANT (2)..(2): Xaa is an amino acid moiety selected from the group consisting of Gly, Ala, Val, Pro, Leu, Ile, Met, Trp and Phe
  VARIANT (3)..(3): Xaa is an amino acid moiety selected from the group consisting of Ser, Thr, Tyr, Cys, Asn, Gln, Lys, Arg, His, a selenocysteinyl moiety, a pyrrolysinyl moiety, Asp and Glu
  VARIANT (4)..(4): Xaa is an acidic amino acid moiety selected from the group consisting of Asp and Glu
  VARIANT (7)..(7): Xaa is an amino acid moiety selected from the group consisting of Ser, Thr, Tyr, Cys, Asn, Gln, Lys, Arg, His, a selenocysteinyl moiety, a pyrrolysinyl moiety, Asp and Glu
  VARIANT (8)..(8): Xaa is an amino acid moiety selected from the group consisting of Gly, Ala, Val, Pro, Leu, Ile, Met, Trp and Phe
  VARIANT (12)..(12): Xaa is an amino acid moiety selected from the group consisting of Gly, Ala, Val, Pro, Leu, Ile, Met, Trp and Phe
  VARIANT (13)..(13): Xaa is an amino acid moiety selected from the group consisting of Ser, Thr, Tyr, Cys, Asn, a selenocysteinyl moiety, a pyrrolysinyl moiety and Gln
  VARIANT (16)..(16): Xaa is an amino acid moiety selected from the group consisting of Gly, Ala, Val, Pro, Leu, Ile, Met, Trp and Phe
  VARIANT (19)..(19): Xaa is an amino acid moiety selected from the group consisting of Gly, Ala, Val, Pro, Leu, Ile, Met, Trp and Phe
  VARIANT (20)..(20): Xaa is an amino acid moiety selected from the group consisting Ala, Gly and Ser
  VARIANT (23)..(23): Xaa is an amino acid moiety selected from the group consisting of Gly, Ala, Val, Pro, Leu, Ile, Met, Trp and Phe
  VARIANT (26)..(26): Xaa is an amino acid moiety selected from the group consisting of Gly, Ala, Val, Pro, Leu, Ile, Met, Trp and Phe
  VARIANT (27)..(27): Xaa is an amino acid moiety selected from the group consisting of Ser, Thr, Tyr, Cys, Asn, a selenocysteinyl moiety, a pyrrolysinyl moiety and Gln
  VARIANT (36)..(36): Xaa can be any naturally occurring amino acid moiety or is a direct bond between the adjacent amino acid moieties
  VARIANT (37)..(37): Xaa is an acidic amino acid moiety selected from the group consisting of Asp and Glu
  VARIANT (38)..(38): Xaa can be any naturally occurring amino acid moiety or is a direct bond between the adjacent amino acid moieties
  VARIANT (39)..(39): Xaa can be any naturally occurring amino acid moiety or is a direct bond between the adjacent amino acid moieties
  VARIANT (44)..(44): Xaa is an amino acid moiety selected from the group consisting of Lys, Arg and His
  VARIANT (45)..(45): Xaa is an amino acid moiety selected from the group consisting of Ser, Thr, Tyr, Cys, Asn, Gln, Lys, Arg, His, a selenocysteinyl moiety, a pyrrolysinyl moiety, Asp and Glu
  VARIANT (47)..(47): Xaa is an amino acid moiety selected from the group consisting of Gly, Ala, Val, Pro, Leu, Ile, Met, Trp and Phe
  VARIANT (51)..(51): Xaa is an amino acid moiety selected from the group consisting of Gly, Ala, Val, Pro, Leu, Ile, Met, Trp and Phe
  VARIANT (53)..(53): Xaa is an amino acid moiety selected from the group consisting of Gly, Ala, Val, Pro, Leu, Ile, Met, Trp and Phe
  VARIANT (54)..(54): Xaa is an amino acid moiety selected from the group consisting of Gly, Ala, Val, Pro, Leu, Ile, Met, Trp and Phe
  VARIANT (56)..(56): Xaa is an amino acid moiety selected from the group consisting of Gly, Ala, Val, Pro, Leu, Ile, Met, Trp and Phe
  VARIANT (58)..(58): Xaa is an amino acid moiety selected from the group consisting of Gly, Ala, Val, Pro, Leu, Ile, Met, Trp and Phe
  VARIANT (58)..(58): Xaa is an amino acid moiety selected from the group consisting of Gly, Ala, Val, Pro, Leu, Ile, Met, Trp and Phe
  VARIANT (59)..(59): Xaa is an amino acid moiety selected from the group consisting of Ser, Thr, Tyr, Cys, Asn, a selenocysteinyl moiety, a pyrrolysinyl moiety and Gln
AXXXTIXXLPTXXEGXPIXXTIXDGXXLGAAIKELXXXXDPLLXXEXEAKXGXXAXNXX

SEQ ID NO: 11 - protein, 61 aa, Clostridium difficile
LPGIHSDLFKTISRPSSIGLDRWEMIKLEAIMKYKKYINNYTSENFDKLDQQLKDNFKLII

SEQ ID NO: 12 - protein, 61 aa, Clostridium difficile
LPGIQPDLFESIEKPSSVTVDFWEMTKLEAIMKYKEYIPEYTSEHFDMLDEEVQSSFESVL

SEQ ID NO: 13 - protein, 31 aa, Clostridium difficile
ANQYEVRINSEGRKELLAHSGKWINKEEAIM

SEQ ID NO: 14 - protein, 31 aa, Clostridium difficile
ANQYEVRINSEGRRELLDHSGEWINKEESII

SEQ ID NO: 15 - protein, 10 aa, Clostridium difficile
GESVYVETEK

SEQ ID NO: 16 - protein, 10 aa, Clostridium difficile
GESIFVETEK

SEQ ID NO: 17 - protein, 18 aa, Clostridium difficile
SVKVQLYAQLFSTGLNTI

SEQ ID NO: 18 - protein, 18 aa, Clostridium difficile
AMKVQVYAQLFSTGLNTI

SEQ ID NO: 19 - protein, 21 aa, Clostridium difficile
LLPIAGISAGIPSLVNNELIL

SEQ ID NO: 20 - protein, 22 aa, Clostridium difficile
LLVPLAGISAGIPSLVNNELVL

SEQ ID NO: 21 - protein, 23 aa, Clostridium difficile
MEGGSGHTVTGNIDHFFSSPSIS

SEQ ID NO: 22 - protein, 23 aa, Clostridium difficile
MEGGSGHTVTDDIDHFFSAPSIT

SEQ ID NO: 23 - protein, 15 aa, Clostridium difficile
PGLRSLENDGTRLLD

SEQ ID NO: 24 - protein, 15 aa, Clostridium difficile
PGLRSLENDGTKLLD

SEQ ID NO: 25 - protein, 59 aa, Clostridium difficile
AVNDTINVLPTITEGIPIVSTILDGINLGAAIKELLDEHDPLLKKELEAKVGVLAINMS

SEQ ID NO: 26 - protein, 59 aa, Clostridium difficile
ALDETIDLLPTLSEGLPIIATIIDGVSLGAAIKELSETSDPLLRQEIEAKIGIMAVNLT

(19) United States
(12) Patent Application Publication
(10) Pub. No.: US 2022/0144924 A1
(43) Pub. Date: May 12, 2022

(54) EPITOPES OF CLOSTRIDIUM DIFFICILE TOXINS A AND B AND USES THEREOF

(71) Applicant: PREVIPHARMA CONSULTING GMBH, Mannheim (DE)

(72) Inventors: Hanne Rieke GERDING, Mannheim (DE); Stephan T. KIESSIG, Wiesloch (DE); Maurice MANDAGO, Schoenau (DE); Marc MAZUR, Weinheim (DE)

(21) Appl. No.: 17/433,743

(22) PCT Filed: Feb. 25, 2020
(86) PCT No.: PCT/EP2020/054879

§ 371 Date: Aug. 25, 2021

(30) Foreign Application Priority Data
Feb. 25, 2019 (EP) 19159044.7

(51) Int. Cl. C07K 16/12; A61K 39/08; A61P 31/04; A61K 9/00; C12N 9/10; C12N 9/16; C12N 11/00; G01N 33/538; G01N 33/573; G01N 33/50

(52) U.S. Cl. CPC C07K 16/1282; A61K 39/08; A61P 31/04; A61K 9/0053; C12N 9/1051; A61K 39/00; C12Y 301/04; C12N 11/00; G01N 33/538; G01N 33/573; G01N 33/5026; C12N 9/16

(57) ABSTRACT

The present invention relates to a polypeptide comprising an epitope having a sequence homology of at least 75% to a sequence section of both Clostridium difficile toxin A and B. Moreover, the present invention refers to a vaccine comprising such polypeptide. The invention further relates to an antibody binding to Clostridium difficile toxins A and B and to a method for isolating and/or detecting such antibody and to uses of the polypeptides and antibodies.

The present invention relates to a polypeptide comprising an epitope having a sequence homology of at least 75% to a sequence section of both Clostridium difficile toxin A and B. Moreover, the present invention refers to a vaccine comprising such polypeptide. The invention further relates to an antibody binding to Clostridium difficile toxins A and B and to a method for isolating and/or detecting such antibody and to uses of the polypeptides and antibodies.

Clostridium difficile-associated disease (CDAD) is a pathologic condition which disrupts the bowel flora of patients causing multiple symptoms ranging from mild to severe. These symptoms include Diarrhea (sometimes bloody), abdominal pain, weight loss, fever, dehydration, fulminant colitis and finally death. In the large intestine, CDAD can particularly arise when the normal flora has been disrupted, e.g. by antibiotic therapy. This disease is caused by an infection of the patient with the ubiquitary, spore-forming, gram-positive bacterium Clostridium difficile.

Clostridium difficile may be reproduced in the intestinal crypts and may release Clostridium difficile toxin A (TcdA) and Clostridium difficile toxin B (TcdB)). This may cause severe inflammation. Mucous and cellular debris may be expelled. This may lead to the formation of pseudo-membranes.

The mechanism of infection is based on the two glycolsyl-transferase toxins, i.e., Clostridium difficile toxins A and B, which are released by the bacterium that proliferates in the anaerobic intestinal crypts. Both toxins can enter the host cells via endocytosis after binding to disaccharide receptors. They typically target and inactivate small GTPases, altering events in the cell ranging from cell signaling to ultrastructure maintenance, finally leading to a cellular intoxication. Both toxins are often highly cytotoxic in low doses, whereas Clostridium difficile toxin B had been found to often be 4 to 200-fold more cytotoxic than Clostridium difficile toxin A, depending on the targeted cell type. It is even capable of triggering apoptosis. In addition, both toxins are capable of modulating inflammatory effects, in combination and alone.

Some hyper-virulent strains are even considered more dangerous due to their production of the binary toxin thus increasing the rate of infection. Clostridium difficile toxin A may attract neutrophils and monocytes. Clostridium difficile toxin B may degrade colonic epithelial cells, both leading to colitis, pseudo-membrane formation and watery diarrhea.

Current treatments include the usage of antibiotics, like metronidazole and vancomycin, as primary options to fight the infection in cases of mild to moderate infections. Severe cases are typically treated by surgical consult in addition to the donation of antibiotics and finally a possible stool transplant. In 2011, Clostridium difficile caused 500,000 severe infections in the U.S. with 29,000 attributable deaths (increased from 14,000 in 2007) making it the most common cause of health care-infections in US hospitals. Severe cases of CDAD, over 10% of all observed cases, were reported to lead directly or indirectly to death. The mortality in severe cases typically increases with the age of the patients, the length of their hospital stays and the usage of antibiotics. Antibiotics are the most important risk factor, since they may disrupt the intestinal flora and permit the toxin-producing Clostridium difficile to establish and proliferate, by killing the bacteria, which then release their spores and toxins. Younger patients suffer mostly from community acquired CDAD. US-A 2018/022784 teaches few examples of Clostridium difficile toxins A and B and antigens which comprise a portion thereof. In its general teaching, US-A 2018/022784 does not specify the length of a polypeptide usable as an antigen.

Immunoglobulins like IgG and IgA have already been proven in their efficacy against a number of other gastrointestinal infections, either caused by bacteria or viruses. Therefore, vaccination would also be desirable for treating or preventing Clostridium difficile-associated infections such as CDAD. Unfortunately, there are no antibiotics with particularly good properties for this purpose available. It is thus desirable to provide further means for vaccination.

Surprisingly, epitopes have been identified that have a high sequence homology to both Clostridium difficile toxins A and B. These are particularly beneficial for generating antibodies that bind to both, Clostridium difficile toxins A and B.

Accordingly, the present invention relates to a polypeptide, comprising at least one epitope of at least one of Clostridium difficile toxin A and Clostridium difficile toxin B, in particular both, or an immunogenic peptidomimetic or retro-inverso polypeptide thereof, wherein said polypeptide is optionally immobilized on a solid support and/or wherein said polypeptide is optionally not more than 100 consecutive amino acids or amino acid analogues in length and the epitope is comprised in both Clostridium difficile toxin A and B.

An aspect of the present invention relates to a polypeptide comprising (or consisting of) at least one epitope, wherein said epitope is at least eight consecutive amino acids in length and said epitope has a sequence homology of at least 75% to a sequence section of Clostridium difficile toxin A and a sequence homology of at least 75% to a sequence section of Clostridium difficile toxin B, and wherein said polypeptide has a (total) length of 8 to 100 consecutive amino acid moieties, or an immunogenic peptidomimetic or retro-inverso polypeptide thereof.

Thus, the person skilled in that art understands that the length of the epitope as well as the length of the total polypeptide which may, optionally, comprise one or more further amino acid moieties in addition to the epitope having at least 75% to a sequence section of Clostridium difficile toxin A and a sequence homology of at least 75% to a sequence section of Clostridium difficile toxin B.

Each polypeptide disclosed herein may be optionally also be an immunogenic peptidomimetic or retro-inverso polypeptide thereof.

The terms “peptidomimetic” and “polypeptide analogue” may be understood interchangeably in the broadest sense as any mimic of a peptide that has similar properties like a peptide, but typically bears higher (biological) stability. Examples for peptidomimetics in the sense of the present invention are such molecular structures partly or completely based on beta amino acid moieties, N-acetylated amino acid moieties (e.g., N-methylated amino acid moieties) and peptoids (i.e., poly-N-substituted glycinyl moieties). Preferably, if the polypeptide is a peptidomimetic, all amino acid moieties of the polypeptide are amino acid analogues of one type (e.g. all are on beta amino acid moieties, all are N-acetylated amino acid moieties or all are N-substituted glycinyl moieties). Likewise, if the polypeptide is a D-peptide analogue, all amino acid moieties of the sequence motifs A1 are D-amino acid moieties.

The term “retro-inverso polypeptide” will be unambiguously understood by those skilled in the art. In a retro-inverso polypeptide, the respective sequence is reversed and D-amino acid moieties are used instead of L-amino acid moieties.

As used herein, the term “immunogenic” may be understood in the broadest sense as being able to trigger an immune response, in particular a humoral immune response. The term “immunogenic” in the context of a polypeptide may be understood in the broadest sense as being able to bind to an antibody.

As used herein, the term “epitope” may be understood in the broadest sense as any antigenic determinant. It may be an antigen or may be part of an antigen that is recognizable by the immune system, sin particular an antibody or antibody fragment.

As used herein, the species names “Clostridium difficile” and “Clostridioides difficile” may be understood interchangeably. The person skilled in the art knows the structure and properties of Clostridium difficile toxins A and B. Typically it is considered that both are composed of four main domains, i.e., GTD (glucosyltransferase domain), CPD (cysteine-protease domain), TD (Translocation domain) and RBD (Receptor-binding domain). Clostridium difficile toxin A typically has a sequence homology of at least 95%, preferably a sequence homology of at least 98%, more preferably a sequence homology of at least 99%, in particular sequence identity to a polypeptide sequence of SEQ ID NO: 1:

MSLISKEELIKLAYSIRPRENEYKTILTNLDEYNKLTTNNNENKYLQLKK
LNESIDVFMNKYKTSSRNRALSNLKKDILKEVILIKNSNTSPVEKNLHFV
WIGGEVSDIALEYIKQWADINAEYNIKLWYDSEAFLVNTLKKAIVESSTT
EALQLLEEEIQNPQFDNMKFYKKRMEFIYDRQKRFINYYKSQINKPTVPT
IDDIIKSHLVSEYNRDETVLESYRTNSLRKINSNHGIDIRANSLFTEQEL
LNIYSQELLNRGNLAAASDIVRLLALKNFGGVYLDVDMLPGIHSDLFKTI
SRPSSIGLDRWEMIKLEAIMKYKKYINNYTSENFDKLDQQLKDNFKLIIE
SKSEKSEIFSKLENLNVSDLEIKIAFALGSVINQALISKQGSYLTNLVIE
QVKNRYQFLNQHLNPAIESDNNFTDTTKIFHDSLFNSATAENSMFLTKIA
PYLQVGFMPEARSTISLSGPGAYASAYYDFINLQENTIEKTLKASDLIEF
KFPENNLSQLTEQEINSLWSFDQASAKYQFEKYVRDYTGGSLSEDNGVDF
NKNTALDKNYLLNNKIPSNNVEEAGSKNYVHYIIQLQGDDISYEATCNLF
SKNPKNSIIIQRNMNESAKSYFLSDDGESILELNKYRIPERLKNKEKVKV
TFIGHGKDEFNTSEFARLSVDSLSNEISSFLDTIKLDISPKNVEVNLLGC
NMFSYDFNVEETYPGKLLLSIMDKITSTLPDVNKNSITIGANQYEVRINS
EGRKELLAHSGKWINKEEAIMSDLSSKEYIFFDSIDNKLKAKSKNIPGLA
SISEDIKTLLLDASVSPDTKFILNNLKLNIESSIGDYIYYEKLEPVKNII
HNSIDDLIDEFNLLENVSDELYELKKLNNLDEKYLISFEDISKNNSTYSV
RFINKSNGESVYVETEKEIFSKYSEHITKEISTIKNSIITDVNGNLLDNI
QLDHTSQVNTLNAAFFIQSLIDYSSNKDVLNDLSTSVKVQLYAQLFSTGL
NTIYDSIQLVNLISNAVNDTINVLPTITEGIPIVSTILDGINLGAAIKEL
LDEHDPLLKKELEAKVGVLAINMSLSIAATVASIVGIGAEVTIFLLPIAG
ISAGIPSLVNNELILHDKATSVVNYFNHLSESKKYGPLKTEDDKILVPID
DLVISEIDFNNNSIKLGTCNILAMEGGSGHTVTGNIDHFFSSPSISSHIP
SLSIYSAIGIETENLDFSKKIMMLPNAPSRVFWWETGAVPGLRSLENDGT
RLLDSIRDLYPGKFYWRFYAFFDYAITTLKPVYEDTNIKIKLDKDTRNFI
MPTITTNEIRNKLSYSFDGAGGTYSLLLSSYPISTNINLSKDDLWIFNID
NEVREISIENGTIKKGKLIKDVLSKIDINKNKLIIGNQTIDFSGDIDNKD
RYIFLTCELDDKISLIIEINLVAKSYSLLLSGDKNYLISNLSNTIEKINT
LGLDSKNIAYNYTDESNNKYFGAISKTSQKSIIHYKKDSKNILEFYNDST
LEFNSKDFIAEDINVFMKDDINTITGKYYVDNNTDKSIDFSISLVSKNQV
KVNGLYLNESVYSSYLDFVKNSDGHHNTSNFMNLFLDNISFWKLFGFENI
NFVIDKYFTLVGKTNLGYVEFICDNNKNIDIYFGEWKTSSSKSTIFSGNG
RNVVVEPIYNPDTGEDISTSLDFSYEPLYGIDRYINKVLIAPDLYTSLIN
INTNYYSNEYYPEIIVLNPNTFHKKVNINLDSSSFEYKWSTEGSDFILVR
YLEESNKKILQKIRIKGILSNTQSFNKMSIDFKDIKKLSLGYIMSNFKSF
NSENELDRDHLGFKIIDNKTYYYDEDSKLVKGLININNSLFYFDPIEFNL
VTGWQTINGKKYYFDINTGAALTSYKIINGKHFYFNNDGVMQLGVFKGPD
GFEYFAPANTQNNNIEGQAIVYQSKFLTLNGKKYYFDNNSKAVTGWRIIN
NEKYYFNPNNAIAAVGLQVIDNNKYYFNPDTAIISKGWQTVNGSRYYFDT
DTAIAFNGYKTIDGKHFYFDSDCVVKIGVFSTSNGFEYFAPANTYNNNIE
GQAIVYQSKFLTLNGKKYYFDNNSKAVTGLQTIDSKKYYFNTNTAEAATG
WQTIDGKKYYFNTNTAEAATGWQTIDGKKYYFNTNTAIASTGYTIINGKH
FYFNTDGIMQIGVFKGPNGFEYFAPANTDANNIEGQAILYQNEFLTLNGK
KYYFGSDSKAVTGWRIINNKKYYFNPNNAIAAIHLCTINNDKYYFSYDGI
LQNGYITIERNNFYFDANNESKMVTGVFKGPNGFEYFAPANTHNNNIEGQ
AIVYQNKFLTLNGKKYYFDNDSKAVTGWQTIDGKKYYFNLNTAEAATGWQ
TIDGKKYYFNLNTAEAATGWQTIDGKKYYFNTNTFIASTGYTSINGKHFY
FNTDGIMQIGVFKGPNGFEYFAPANTDANNIEGQAILYQNKFLTLNGKKY
YFGSDSKAVTGLRTIDGKKYYFNTNTAVAVTGWQTINGKKYYFNTNTSIA
STGYTIISGKHFYFNTDGIMQIGVFKGPDGFEYFAPANTDANNIEGQAIR
YQNRFLYLHDNIYYFGNNSKAATGWVTIDGNRYYFEPNTAMGANGYKTID
NKNFYFRNGLPQIGVFKGSNGFEYFAPANTDANNIEGQAIRYQNRFLHLL
GKIYYFGNNSKAVTGWQTINGKVYYFMPDTAMAAAGGLFEIDGVIYFFGV
DGVKAPGIYG

Clostridium difficile toxin B typically has a sequence homology of at least 95%, preferably a sequence homology of at least 98%, more preferably a sequence homology of at least 99%, in particular sequence identity to a polypeptide sequence of SEQ ID NO: 2:

MSLVNRKQLEKMANVRFRTQEDEYVAILDALEEYHNMSENTVVEKYLKLK
DINSLTDIYIDTYKKSGRNKALKKFKEYLVTEVLELKNNNLTPVEKNLHF
VWIGGQINDTAINYINQWKDVNSDYNVNVFYDSNAFLINTLKKTVVESAI
NDTLESFRENLNDPRFDYNKFFRKRMEIIYDKQKNFINYYKAQREENPEL
IIDDIVKTYLSNEYSKEIDELNTYIEESLNKITQNSGNDVRNFEEFKNGE
SFNLYEQELVERWNLAAASDILRISALKEIGGMYLDVDMLPGIQPDLFES
IEKPSSVTVDFWEMTKLEAIMKYKEYIPEYTSEHFDMLDEEVQSSFESVL
ASKSDKSEIFSSLGDMEASPLEVKIAFNSKGIINQGLISVKDSYCSNLIV
KQIENRYKILNNSLNPAISEDNDFNTTTNTFIDSIMAEANADNGRFMMEL
GKYLRVGFFPDVKTTINLSGPEAYAAAYQDLLMFKEGSMNIHLIEADLRN
FEISKTNISQSTEQEMASLWSFDDARAKAQFEEYKRNYFEGSLGEDDNLD
FSQNIVVDKEYLLEKISSLARSSERGYIHYIVQLQGDKISYEAACNLFAK
TPYDSVLFQKNIEDSEIAYYYNPGDGEIQEIDKYKIPSIISDRPKIKLTF
IGHGKDEFNTDIFAGFDVDSLSTEIEAAIDLAKEDISPKSIEINLLGCNM
FSYSINVEETYPGKLLLKVKDKISELMPSISQDSIIVSANQYEVRINSEG
RRELLDHSGEWINKEESIIKDISSKEYISFNPKENKITVKSKNLPELSTL
LQEIRNNSNSSDIELEEKVMLTECEINVISNIDTQIVEERIEEAKNLTSD
SINYIKDEFKLIESISDALCDLKQQNELEDSHFISFEDISETDEGFSIRF
INKETGESIFVETEKTIFSEYANHITEEISKIKGTIFDTVNGKLVKKVNL
DTTHEVNTLNAAFFIQSLIEYNSSKESLSNLSVAMKVQVYAQLFSTGLNT
ITDAAKVVELVSTALDETIDLLPTLSEGLPIIATIIDGVSLGAAIKELSE
TSDPLLRQEIEAKIGIMAVNLTTATTAIITSSLGIASGFSILLVPLAGIS
AGIPSLVNNELVLRDKATKVVDYFKHVSLVETEGVFTLLDDKIMMPQDDL
VISEIDFNNNSIVLGKCEIWRMEGGSGHTVTDDIDHFFSAPSITYREPHL
SIYDVLEVQKEELDLSKDLMVLPNAPNRVFAWETGWTPGLRSLENDGTKL
LDRIRDNYEGEFYWRYFAFIADALITTLKPRYEDTNIRINLDSNTRSFIV
PIITTEYIREKLSYSFYGSGGTYALSLSQYNMGINIELSESDVWIIDVDN
VVRDVTIESDKIKKGDLIEGILSTLSIEENKIILNSHEINFSGEVNGSNG
FVSLTFSILEGINAIIEVDLLSKSYKLLISGELKILMLNSNHIQQKIDYI
GFNSELQKNIPYSFVDSEGKENGFINGSTKEGLFVSELPDVVLISKVYMD
DSKPSFGYYSNNLKDVKVITKDNVNILTGYYLKDDIKISLSLTLQDEKTI
KLNSVHLDESGVAEILKFMNRKGNTNTSDSLMSFLESMNIKSIFVNFLQS
NIKFILDANFIISGTTSIGQFEFICDENDNIQPYFIKFNTLETNYTLYVG
NRQNMIVEPNYDLDDSGDISSTVINFSQKYLYGIDSCVNKVVISPNIYTD
EINITPVYETNNTYPEVIVLDANYINEKINVNINDLSIRYVWSNDGNDFI
LMSTSEENKVSQVKIRFVNVFKDKTLANKLSFNFSDKQDVPVSEIILSFT
PSYYEDGLIGYDLGLVSLYNEKFYINNFGMMVSGLIYINDSLYYFKPPVN
NLITGFVTVGDDKYYFNPINGGAASIGETIIDDKNYYFNQSGVLQTGVFS
TEDGFKYFAPANTLDENLEGEAIDFTGKLIIDENIYYFDDNYRGAVEWKE
LDGEMHYFSPETGKAFKGLNQIGDYKYYFNSDGVMQKGFVSINDNKHYFD
DSGVMKVGYTEIDGKHFYFAENGEMQIGVFNTEDGFKYFAHHNEDLGNEE
GEEISYSGILNFNNKIYYFDDSFTAVVGWKDLEDGSKYYFDEDTAEAYIG
LSLINDGQYYFNDDGIMQVGFVTINDKVFYFSDSGIIESGVQNIDDNYFY
IDDNGIVQIGVFDTSDGYKYFAPANTVNDNIYGQAVEYSGLVRVGEDVYY
FGETYTIETGWIYDMENESDKYYFNPETKKACKGINLIDDIKYYFDEKGI
MRTGLISFENNNYYFNENGEMQFGYINIEDKMFYFGEDGVMQIGVFNTPD
GFKYFAHQNTLDENFEGESINYTGWLDLDEKRYYFTDEYIAATGSVIIDG
EEYYFDPDTAQLVISE

In a further preferred embodiment, the epitope comprises (or consist of) a polypeptide sequence of at least eight consecutive amino acid moieties in length having a sequence homology of at least 75% to SEQ ID NO: 1 or 2 or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 1 or 2 or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 1 or 2, wherein not more than two amino acid moieties have been deleted or replaced, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 1 or 2, wherein (exactly) one amino acid moiety has been deleted or replaced, in particular has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 1 or 2, wherein (exactly) two amino acid moiety have been deleted or replaced, in particular have been replaced by analogue amino acid moieties, or an immunogenic peptidomimetic or retro-inverso polypeptide thereof.

Such polypeptide or immunogenic peptidomimetic or retro-inverso thereof may also be between 8 and 100, between 9 and 90, between 10 and 80, between 11 and 70, between 18 and 50, between 9 and 40, between 8 and 20, between 12 and 25, between 13 and 20, between 14 and 18 consecutive amino acid moieties (or analogues thereof) in length.

In a further preferred embodiment, the epitope comprises (or consist of) a polypeptide sequence having a sequence homology of at least 80%, at least 85%, at least 90%, at least 95%, or at least 98%, or identity to SEQ ID NO: 1 and/or 2.

As used throughout the present invention, the term “replaced by an analogue amino acid moiety” may be understood in the broadest sense as being substituted by an amino acid moiety of similar physicochemical properties.

For example, “replaced by an analogue amino acid moiety” may have the meaning that a non-polar amino acid moiety may be substituted by another non-polar amino acid moiety, in particular an aliphatic non-polar amino acid moiety may be substituted by another aliphatic non-polar amino acid moiety and an aromatic non-polar amino acid moiety may be substituted by another aromatic non-polar amino acid moiety. In this context, a non-polar amino acid moiety may be selected from the group consisting of G (Gly), A (Ala), V (Val), P (Pro), L (Leu), I (Ile), M (Met), W (Trp) and F (Phe). An aliphatic non-polar amino acid moiety may be selected from the group consisting of G (Gly), A (Ala), V (Val), P (Pro), L (Leu), I (Ile), and M (Met). An aromatic non-polar amino acid moiety may be selected from the group consisting of W (Trp) and F (Phe). In a preferred embodiment, in particular G (Gly), A (Ala), V (Val), L (Leu) or I (Ile) may be replaced by one another.

For example, “replaced by an analogue amino acid moiety” may also have the meaning that a polar (uncharged) amino acid moiety may be substituted by another polar (uncharged) amino acid moiety, in particular an aliphatic polar (uncharged) amino acid moiety may be substituted by another aliphatic polar (uncharged) amino acid moiety. In this context, a polar (uncharged) amino acid moiety may be selected from the group consisting of S (Ser), T (Thr), Y (Tyr), C (Cys), N (Asn), U (Sec, selenocysteinyl), O (Pyl, pyrrolysinyl) and Q (Gln). An aliphatic polar (uncharged) amino acid moiety may be selected from the group consisting of S (Ser), T (Thr), C (Cys), N (Asn), U (Sec), O (Pyl) and Q (Gln). In a preferred embodiment, in particular S (Ser) may be replaced by T (Thr) and vice versa and Q (Gln) may be replaced by N (Asn) and vice versa.

For example, “replaced by an analogue amino acid moiety” may also have the meaning that a basic amino acid moiety may be substituted by another basic amino acid moiety.

In this context, a basic amino acid moiety may be selected from the group consisting of K (Lys), R (Arg) and H (His). In a preferred embodiment, in particular K (Lys) may be replaced by R (Arg) and vice versa.

For example, “replaced by an analogue amino acid moiety” may also have the meaning that an acidic amino acid moiety may be substituted by another acidic amino acid moiety. In this context, an acidic amino acid moiety may be selected from the group consisting of D (Asp) and E (Glu).

For example, “replaced by an analogue amino acid moiety” may also have the meaning that a polar amino acid moiety including interactions of the opposite charge may be substituted comparable amino acid moieties. Such amino acid moieties which are exchangeable by one another may be selected from the group consisting of S (Ser), T (Thr), Y (Tyr), C (Cys), N (Asn), Q (Gln), K (Lys), R (Arg), H (His), U (Sec), O (Pyl), D (Asp) and E (Glu).

For example, “replaced by an analogue amino acid moiety” may also have the meaning that a small-sized amino acid moiety may be replaced by another small-sized amino acid moiety. In this context, a small-sized amino acid moiety may be selected from the group consisting of A (Ala), G (Gly) and S (Ser).

For example, “replaced by an analogue amino acid moiety” may also have the meaning that an at least partly polar amino acid moiety may be substituted by another at least partly polar amino acid moiety, in particular an aliphatic at least partly polar amino acid moiety may be substituted by another aliphatic at least partly polar amino acid moiety and an aromatic at least partly polar amino acid moiety may be substituted by another aromatic at least partly polar amino acid moiety. In this context, an at least partly polar amino acid moiety may be selected from the group consisting of Y (Tyr), G (Gly), A (Ala), V (Val), P (Pro), L (Leu), I (Ile), M (Met), W (Trp) and F (Phe). An aliphatic at least partly polar amino acid moiety may be selected from the group consisting of G (Gly), A (Ala), V (Val), P (Pro), L (Leu), I (Ile) and M (Met). An aromatic at least partly polar amino acid moiety may be selected from the group consisting of Y (Tyr), W (Trp) and F (Phe).

In a preferred embodiment, the epitope is between 8 and 100, between 8 and 61, between 8 and 50, between 8 and 40, between 8 and 30, between 8 and 20, between 8 and 17, between 8 and 11, consecutive amino acids in length. For example, an epitope is 8, 9, 10, 11, 12, 13, 14, 15, 16, 17, 18, 19 or 20 consecutive amino acids in length.

In a preferred embodiment, the polypeptide is between 8 and 100, between 8 and 61, between 8 and 50, between 8 and 40, between 8 and 30, between 8 and 20, between 8 and 17, between 8 and 11, consecutive amino acids in length. For example, an epitope is 8, 9, 10, 11, 12, 13, 14, 15, 16, 17, 18, 19 or 20 consecutive amino acids in length.

In a preferred embodiment, the epitope has a sequence homology of at least 80%, at least 85%, at least 90%, at least 95%, at least 98%, at least 99%, or sequence identity to a sequence section of Clostridium difficile toxin A.

In a preferred embodiment, the epitope has a sequence homology of at least 80%, at least 85%, at least 90%, at least 95%, at least 98%, at least 99%, or sequence identity to a sequence section of Clostridium difficile toxin B.

In a preferred embodiment, the epitope has a sequence homology of at least 80%, at least 85%, at least 90%, at least 95%, at least 98%, at least 99%, or sequence identity to a sequence section of both Clostridium difficile toxin A and B.

The polypeptides (i.e., the peptidic structures, peptidic) may be each independently from another be obtained by any means know for this purpose in the art.

In a preferred embodiment, the polypeptide is obtained by means selected from the group consisting of polypeptide synthesis, gene technological means, and isolation of Clostridium difficile toxin A or Clostridium difficile toxin B and subsequent protein digestion.

Preferably, the polypeptides are obtained by solid phase peptide synthesis (SPPS) such as of Fmoc- or Boc-based SPPS. Alternatively, the polypeptides may also be obtained by liquid phase peptide synthesis (LPPS) or, in the case of consisting of L-amino acid moieties, by means of biotechnology means such as heterologous expression in a genetically modified organism excluding human bodies such as, e.g., bacteria (e.g., Clostridium difficile, E. coli), fungi (e.g., yeast), mammalian cells or mammalians excluding humans, insect cells or insects, plant cells or plants, etc. Accordingly, genetic manipulation of a host organism with the sequence comprising an epitope of the present invention being genetically fused to a gene encoding for the rest of the polypeptide may be used.

Gene technological means includes, for instance, recombinant expression (e.g., heterologous expression and/or overexpression).

In a preferred embodiment, the epitope has a sequence homology of at least 75% to a sequence section located at the outer surface of Clostridium difficile toxin A and has a sequence homology of at least 75% to a sequence section located at the outer surface of Clostridium difficile toxin B.

In a preferred embodiment, the epitope has (accordingly also: consists of, or is, respectively) or comprises an amino acid sequence selected from the group consisting of

(SEQ ID NO: 3)
ANQYEVRINSEGRX²³ELLX¹HSGX²⁵WINKEEX²⁶IX²¹;
(SEQ ID NO: 4)
GESX²¹X²⁷VETEK;
(SEQ ID NO: 5)
X²⁶X²¹KVQX²¹YAQLFSTGLNTI;
(SEQ ID NO: 6)
LX²¹PX²¹AGISAGIPSLVNNELX²¹L;
(SEQ ID NO: 7)
DDLVISEIDFNNNSI;
(SEQ ID NO: 8)
MEGGSGHTVTX¹X²⁵IDHFFSX²⁶PSIX²²;
(SEQ ID NO: 9)
PGLRSLENDGTX²³LLD;
and
(SEQ ID NO: 10)
AX²¹X²⁵X²⁴TIX²⁵X²¹LPTX²¹X²²EGX²¹PIX²¹X²⁶TIX²¹DGX²¹
X²²LGAAIKELX¹X²⁴X¹X¹DPLLX²³X²⁵EX²¹EAKX²¹GX²¹X²¹A
X²¹NX²¹X²²,

wherein:
X¹ is any naturally occurring amino acid moiety or is a direct bond between the adjacent amino acid moieties;
X²¹ is an amino acid moiety selected from the group consisting of G (Gly), A (Ala), V (Val), P (Pro), L (Leu), I (Ile), M (Met), W (Trp) and F (Phe);
X²²: is an amino acid moiety selected from the group consisting of S (Ser), T (Thr), Y (Tyr), C (Cys), N (Asn), U (Sec, selenocysteinyl), 0 (Pyl, pyrrolysinyl) and Q (Gln);
X²³ is an amino acid moiety selected from the group consisting of K (Lys), R (Arg) and H (His);
X²⁴ is an acidic amino acid moiety selected from the group consisting of D (Asp) and E (Glu);
X²⁵ is an amino acid moiety selected from the group consisting of S (Ser), T (Thr), Y (Tyr), C (Cys), N (Asn), Q (Gln), K (Lys), R (Arg), H (His), U (Sec, selenocysteinyl), O (Pyl, pyrrolysinyl), D (Asp) and E (Glu);
X²⁶ is an amino acid moiety selected from the group consisting A (Ala), G (Gly) and S (Ser); and
X²⁷ is an amino acid moiety selected from the group consisting of Y (Tyr), G (Gly), A (Ala), V (Val), P (Pro), L (Leu), I (Ile), M (Met), W (Trp) and F (Phe),
or is an immunogenic peptidomimetic or retro-inverso polypeptide thereof.

As used in the context of the present invention, the common abbreviations for amino acid moieties are used.

In a further preferred embodiment, the epitope comprises (or consist of) a polypeptide sequence of at least eight consecutive amino acid moieties in length of any of SEQ ID NO: 3, 4, 5, 6, 7, 8, 9 or 10 or an immunogenic peptidomimetic or retro-inverso polypeptide thereof. In a further preferred embodiment, the epitope comprises (or consist of) a polypeptide sequence of at least nine, at least ten, at least eleven, at least twelve, at least 13, or at least 14 consecutive amino acid moieties in length of any of SEQ ID NO: 3, 4, 5, 6, 7, 8, 9 or 10 or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein not more than two amino acid moieties have been deleted or replaced in comparison to the sequence having the largest homology of SEQ ID NO: 1 or 2 in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein not more than two amino acid moieties have been deleted or replaced in comparison to the sequence having the largest homology of SEQ ID NO: 1 and wherein not more than two amino acid moieties have been deleted or replaced in comparison to the sequence having the largest homology of SEQ ID NO: 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein (exactly) one amino acid moiety has been deleted or replaced in comparison to the sequence having the largest homology of SEQ ID NO: 1 or 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein the sequence has a sequence section of SEQ ID NO: 1 and wherein not more than two amino acid moieties have been deleted or replaced in comparison to the sequence having the largest homology of SEQ ID NO: 2, in particular, if replaced, each have been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein the sequence has a sequence section of SEQ ID NO: 2 and wherein not more than two amino acid moieties have been deleted or replaced in comparison to the sequence having the largest homology of SEQ ID NO: 1, in particular, if replaced, each have been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein (exactly) two amino acid moieties have been deleted or replaced in comparison to the sequence having the largest homology of SEQ ID NO: 1 or 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 3, 4, 5, 6, 7, 8, 9 or 10, wherein no, one or two amino acid moieties have been depleted of replaced, in particular by a homologue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length having a sequence homology of at least 75% to a sequence to a sequence selected from the group consisting of:

(SEQ ID NO: 11)
LPGIHSDLFKTISRPSSIGLDRWEMIKLEAIMKYKKYINNYTSENFDKLD
QQLKDNFKLII,
and
(SEQ ID NO: 12)
LPGIQPDLFESIEKPSSVTVDFWEMTKLEAIMKYKEYIPEYTSEHFDMLD
EEVQSSFESVL,

or an immunogenic peptidomimetic or retro-inverso polypeptide thereof.

In a further preferred embodiment, the polypeptide or immunogenic peptidomimetic or retro-inverso peptide of the present invention (in particular such of SEQ ID NO: 11 or 12) thereof may also be between 8 and 61, between 9 and 50, between 10 and 40, between 11 and 30, between 12 and 25, between 13 and 20, between 14 and 18 consecutive amino acid moieties (or analogues thereof) in length.

In a further preferred embodiment, the epitope comprises (or consist of) a polypeptide sequence having a sequence homology of at least 80%, at least 85%, at least 90%, at least 95%, or at least 98%, or identity to SEQ ID NO: 11 and/or 12.

In a further preferred embodiment, the epitope comprises (or consist of) a polypeptide sequence of at least eight consecutive amino acid moieties in length of SEQ ID NO: 11 or 12 or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 11 or 12, wherein not more than two amino acid moieties have been deleted or replaced, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 11 or 12, wherein (exactly) one amino acid moiety has been deleted or replaced, in particular has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of SEQ ID NO: 11 or 12, wherein (exactly) two amino acid moiety have been deleted or replaced, in particular have been replaced by analogue amino acid moieties, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 11 or 12, wherein not more than two amino acid moieties have been deleted or replaced in comparison to the sequence having the largest homology of SEQ ID NO: 1 and wherein not more than two amino acid moieties have been deleted or replaced in comparison to the sequence having the largest homology of SEQ ID NO: 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NO: 11 or 12, wherein (exactly) one amino acid moiety has been deleted or replaced in comparison to the sequence having the largest homology of SEQ ID NO: 1 or 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length having a sequence homology of at least 75% to a sequence to a sequence selected from the group consisting of:

(SEQ ID NO: 13)
ANQYEVRINSEGRKELLAHSGKWINKEEAIM;
(SEQ ID NO: 14)
ANQYEVRINSEGRRELLDHSGEWINKEESII;
(SEQ ID NO: 15)
GESVYVETEK;
(SEQ ID NO: 16)
GESIFVETEK;
(SEQ ID NO: 17)
SVKVQLYAQLFSTGLNTI;
(SEQ ID NO: 18)
AMKVQVYAQLFSTGLNTI;
(SEQ ID NO: 19)
LLPIAGISAGIPSLVNNELIL;
(SEQ ID NO: 20)
LLVPLAGISAGIPSLVNNELVL;
(SEQ ID NO: 21)
MEGGSGHTVTGNIDHFFSSPSIS;
(SEQ ID NO: 22)
MEGGSGHTVTDDIDHFFSAPSIT;
(SEQ ID NO: 23)
PGLRSLENDGTRLLD;
(SEQ ID NO: 24)
PGLRSLENDGTKLLD;
(SEQ ID NO: 25)
AVNDTINVLPTITEGIPIVSTILDGINLGAAIKELLDEHDPLLKKELEAK
VGVLAINMS;
and
(SEQ ID NO: 26)
ALDETIDLLPTLSEGLPIIATIIDGVSLGAAIKELSETSDPLLRQEIEAK
IGIMAVNLT.

In a further preferred embodiment, the polypeptide or immunogenic peptidomimetic or retro-inverso thereof of the present invention of any of SEQ ID NOs: 13 to 26 may be between 8 and 59, between 9 and 50, between 10 and 40, between 10 and 30, between 10 and 25, between 10 and 20, between 10 and 18 consecutive amino acid moieties (or analogues thereof) in length.

In a further preferred embodiment, the epitope comprises (or consist of) a polypeptide sequence having a sequence homology of at least 80%, at least 85%, at least 90%, at least 95%, or at least 98%, or identity to any of SEQ ID NOs: 13 to 24.

In a further preferred embodiment, the epitope comprises (or consist of) a polypeptide sequence of at least eight consecutive amino acid moieties in length of any of SEQ ID NOs: 13 to 26, wherein not more than two amino acid moieties have been deleted or replaced, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NOs: 13 to 26, wherein (exactly) one amino acid moiety has been deleted or replaced, in particular has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NOs: 13 to 26, wherein (exactly) two amino acid moiety have been deleted or replaced, in particular have been replaced by analogue amino acid moieties, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NOs: 13 to 26, wherein not more than two amino acid moieties have been deleted or replaced in comparison to the sequence having the largest homology of SEQ ID NO: 1 or 2 in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NOs: 13 to 26, wherein not more than two amino acid moieties have been deleted or replaced in comparison to the sequence having the largest homology of SEQ ID NO: 1 and wherein not more than two amino acid moieties have been deleted or replaced in comparison to the sequence having the largest homology of SEQ ID NO: 2, in particular, if replaced, each has been replaced by an analogue amino acid moiety, or an immunogenic peptidomimetic or retro-inverso polypeptide thereof.

In a further preferred embodiment, the epitope comprises (or consist of) a polypeptide sequence of at least eight consecutive amino acid moieties in length of any of SEQ ID NOs: 13 to 26, wherein (exactly) one amino acid moiety has been deleted or replaced in comparison to the sequence having the largest homology of SEQ ID NO: 1 or 2, in particular, if replaced, each has been replaced by an analogue amino acid moiety, or an immunogenic peptidomimetic or retro-inverso polypeptide thereof.

The polypeptide may be an unbound polypeptide or may be bound to one or more other chemical entities.

In a preferred embodiment, the polypeptide is immobilized on a solid support.

In a preferred embodiment, the solid support is a solid phase of an affinity column. In another preferred embodiment, such solid support is a surface of a microtiter plate.

The present invention further relates to a mixture of at least two polypeptides of the present, in particular a mixture of at least one polypeptide comprising (or consisting of) at least one epitope of at least one of Clostridium difficile toxin A and of at least one polypeptide comprising (or consisting of) at least one epitope of at least one of Clostridium difficile toxin B or a mixture of at least two polypeptides comprising (or consisting of) each at least one epitope of at least one of Clostridium difficile toxin A or a mixture of at least two polypeptides comprising (or consisting of) each at least one epitope of at least one of Clostridium difficile toxin B, wherein each of the polypeptides may optionally also be replaced by an immunogenic peptidomimetic thereof or an immunogenic retro-inverso polypeptide thereof, wherein said polypeptide is immobilized on a solid support.

A further aspect of the present invention relates to a vaccine comprising (or consisting of) at least one polypeptide of the present invention and at least one pharmaceutically acceptable carrier.

It will be understood that all definitions are preferred embodiments as indicated above mutatis mutandis also apply to the vaccine.

As used herein, the terms “pharmaceutically acceptable carrier”, “pharmaceutically acceptable excipient”, “carrier” and “excipient” may be understood interchangeably in the broadest sense as any substance that may support the pharmacological acceptance of the vaccine.

In a preferred embodiment, vaccine prepared for final administration enables routes of administration which circumvent the first pass effect. More preferably, the vaccine is prepared to be suitable for administration by injection into the patient (e.g., suitable for administration routes selected from the group consisting of intravenous (i.v.), intraarterial (i.a.), intraperitoneal (i.p.), intramuscular (i.m.), and subcutaneous (s.c.) injection). Alternatively or additionally, the vaccine may also be suitable for other routes of administration such as, e.g., nasal or transdermal administration.

The vaccine ready to use preferably is a liquid formulation, in particular an injection portion. The storage form may also be liquid, but may also be a dried form (e.g. a powder such as a powder comprising dried or freeze-dried one or more polypeptides of the present invention) or may be a paste or syrup or the like. Optionally, a dried form, paste or syrup may be dissolved or emulsified prior to being administered to the patient.

A pharmaceutically acceptable carrier may exemplarily be selected from the list consisting of an aqueous buffer, saline, water, dimethyl sulfoxide (DMSO), ethanol, vegetable oil, paraffin oil or combinations of two or more thereof. Furthermore, the pharmaceutically acceptable carrier may optionally contain one or more detergent(s), one or more foaming agent(s) (e.g., sodium lauryl sulfate (SLS), sodium doceyl sulfate (SDS)), one or more coloring agent(s) (e.g., food coloring), one or more vitamin(s), one or more salt(s) (e.g., sodium, potassium, calcium, zinc salts), one or more humectant(s) (e.g., sorbitol, glycerol, mannitol, propylenglycol, polydextrose), one or more enzyme(s), one or more preserving agent(s) (e.g., benzoic acid, methylparabene, one or more antioxidant(s), one or more herbal and plant extract(s), one or more stabilizing agent(s), one or more chelating agents (e.g., ethylenediaminetetraacetic acid (EDTA), and/or one or more uptake mediator(s) (e.g., polyethylene imine (PEI), a cell-penetrating peptide (CPP), a protein transduction domain (PTD), an antimicrobial peptide, etc.).

The present invention also relates to a dosage unit of the vaccine of the present invention. Exemplarily, the present invention may refer to a single dose container or to a multiple dosage form.

A still further aspect of the present invention refers to the vaccine of the present invention for use in a method for preventing an individual from developing a Clostridium difficile infection.

It will be understood that all definitions are preferred embodiments as indicated above mutatis mutandis also apply to the vaccine for use.

In other words, the present invention also relates to a method for preventing an individual from developing a Clostridium difficile infection in a patient, wherein said patient is administered with a sufficient amount of the vaccine of the present invention. Accordingly, a sill further aspect of the present invention relates to the use of a vaccine of the present invention for vaccination.

As used in the context of the present invention, the term “patient” may be understood in the broadest sense as any living being, which is preferably any animal, more preferably a mammal including human, in particular a human being.

Preferably, administration is systemic administration (e.g., intravenously (i.v.), intraarterially (i.a.), intraperitoneally (i.p.), intramusculary (i.m.), subcutaneously (s.c.), transdermally, nasally), intradermally (i.d.). Alternatively, administration may also be local administration (e.g., intrathecally or intravitreally). Preferably, administration is systemic administration, in particular intravenous injection.

It will be understood that the vaccine may trigger an immune response, in particular a humoral immune response, i.e., the generation of antibodies.

Accordingly, a still further aspect of the present invention relates to an antibody or antibody fragment binding to Clostridium difficile toxin A with a dissociation constant Kd of less than 100 nM and to Clostridium difficile toxin B with a dissociation constant Kd of less than 100 nM.

It will be understood that all definitions are preferred embodiments as indicated above mutatis mutandis also apply to the antibody or antibody fragment

An antibody may be an antibody of any antibody class such as, e.g., IgG, IgA, IgD, IgM or IgE. An antibody fragment is preferably a fragment antigen-binding (Fab). An antibody or antibody fragment may also be an antibody mimetic such as, e.g., a designed ankyrin repeat protein (DARPin), an affibody, an affilins, an affimer, an affitins, an alphabodies, an anticalins, an avimerm, a fynomer, a Kunitz domain peptide, a monobody or a nanoCLAMP.

In a preferred embodiment, the antibody or antibody fragment is a neutralizing antibody or antibody fragment which may decrease enzymatic activity of its target (i.e, Clostridium difficile toxin A or B or, in particular Clostridium difficile toxins A and B) and/or inhibiting its capability to bind onto the receptors.

In a preferred embodiment, the antibody or antibody fragment binds to Clostridium difficile toxin A with a dissociation constant Kd of less than 50 nM, less than 25 nM or less than 10 nM.

In a preferred embodiment, the antibody or antibody fragment binds to Clostridium difficile toxin B with a dissociation constant Kd of less than 50 nM, less than 25 nM or less than 10 nM.

In a preferred embodiment, the antibody or antibody fragment binds to both Clostridium difficile toxin A and B each with a dissociation constant Kd of less than 50 nM, less than 25 nM or less than 10 nM.

In a preferred embodiment, the antibody or antibody fragment binds to an epitope as defined herein with a dissociation constant Kd of less than 100 nM. In a preferred embodiment, the antibody or antibody fragment binds to an epitope as defined herein with a dissociation constant Kd of less than 50 nM, less than 25 nM or less than 10 nM. In a preferred embodiment, antibody or antibody fragment of the present invention is isolated out of general antibody pools (e.g., IgA pools) using positive affinity chromatography. The ligands on the affinity resin may be immunogenic epitopes of the Clostridium difficile toxins A and B, which allow the binding of specific antibodies or antibody fragments (e.g., IgAs).

The antibody or antibody fragment of the present invention may be used for any purpose, including in vivo and in vitro uses.

Such antibody or antibody fragment may also be isolated from any fluid, in particular from body fluids such as blood or fractions thereof. This may be performed for preparative or analytical purposes.

Further, such antibody or antibody fragment may also be detected in any fluid, in particular in body fluids such as blood or fractions thereof. This may be performed for diagnostic or scientific purposes.

A further aspect of the present invention refers to a method for isolating and/or detecting an antibody or antibody fragment binding to Clostridium difficile toxins A and B from a fluid containing the antibody or antibody fragment, wherein said method comprises the following steps:
(i) providing:
   the fluid containing the antibody or antibody fragment, and
   a polypeptide of the present invention immobilized on a solid support;
(ii) contacting the fluid with the immobilized polypeptide and allowing the antibody or antibody fragment to bind to the immobilized polypeptide; and
(iii) removing at least parts of the unbound fluid and optionally washing the solid support with a fluid not containing the containing the antibody or antibody fragment.

It will be understood that all definitions are preferred embodiments as indicated above mutatis mutandis also apply to the method. This method preferably is an in vitro method.

In a preferred embodiment, the antibody or antibody fragment binding to Clostridium difficile toxins A and B is an antibody or antibody fragment of the present invention. Therefore, in a preferred embodiment, the method is a method for isolating and/or detecting an antibody or antibody fragment of the present invention.

In a preferred embodiment, the fluid is a body fluid. In a preferred embodiment, the body fluid selected from the group consisting of blood plasma and a fraction of blood plasma. In a preferred embodiment, the method further comprises preparing of a fraction of blood plasma by means of a Cohn or Kistler-Nitschmann process.

In a further preferred embodiment, the fluid is a supernatant or extracellular liquid from a cell culture such as, e.g., from a cell line expressing such antibody or antibody fragment. For example, such cell culture may be a hybridoma cell culture.

The polypeptide of the present invention may be immobilized on a solid support by any means. It may be directly conjugated to the surface or may be conjugated via any linker.

In a preferred embodiment, the solid support is a solid phase of an affinity column removing at least parts of the unbound fluid also includes the removal of unbound antibodies and antibody fragments having no or low binding affinity to the polypeptide of the present invention including the at least one epitope.

The present invention also deals with the purification of immunoglobulins with varying degrees of affinity, including immunoglobulins with neutralizing capabilities, against bacterial toxins from Clostridium difficile and against bacteria like Clostridium difficile and viruses and their affiliated proteins including their toxins and subtypes.

In a preferred embodiment, the present invention relates to a method for isolating an antibody or antibody fragment binding to an epitope of at least one of Clostridium difficile toxin A and Clostridium difficile toxin B, in particular an antibody or antibody fragment of the present invention, from a body fluid, wherein said method comprises the following consecutive steps:
(a-i) providing:
   the body fluid, and
   a polypeptide according to any of the present invention or a mixture of polypeptides of the present invention immobilized on the solid phase of an affinity column;
(a-ii) contacting the body fluid with the immobilized polypeptide;
(a-iii) eluting the unbound body fluid and optionally washing the solid support by a flow-through of a buffer through the column; and
(a-iv) eluting the bound antibody or antibody fragment from the affinity column.

In another preferred embodiment, the present invention relates to a method for detecting an antibody or antibody fragment binding to an epitope of at least one of Clostridium difficile toxin A and Clostridium difficile toxin B, in particular an antibody or antibody fragment of the present invention, in a body fluid, wherein said method comprises the following consecutive steps:
(b-i) providing:
   the body fluid, and
   a polypeptide of the present invention immobilized on a solid support;
(b-ii) contacting the body fluid with the immobilized polypeptide;
(b-iii) removing the unbound body fluid and optionally washing the solid support; and
(b-iv) detecting the bound antibody or antibody fragment.

In a preferred embodiment, the method is performed by means of affinity chromatography and said method comprises:
(i-c) providing:
   blood plasma or a fraction thereof, and
   a polypeptide or a mixture of the present invention immobilized on the solid phase of an affinity column;
(ii-c) contacting the blood plasma with the immobilized polypeptide;
(iii-c) eluting the unbound blood plasma or a fraction thereof and optionally washing the solid support by a flow-through of a buffer through the column; and
(iv-c) eluting the bound antibody or antibody fragment of the present invention from the affinity column and detecting said antibody.

In a preferred embodiment, the method is performed by means of an enzyme-linked immunosorbent assay (ELISA) and said method comprises:
(i-d) providing:
   blood plasma or a fraction thereof, and
   a polypeptide or a mixture of polypeptides of the present invention immobilized on a solid phase;
(ii-d) contacting the blood plasma with the immobilized polypeptide;
(iii-d) removing the unbound blood plasma or a fraction thereof and optionally washing the solid support with a buffer; and
(iv-d) detecting the bound antibody or antibody fragment with a secondary antibody selectively to the Fc part of the antibody or antibody fragment of the present invention and detecting said secondary antibody, wherein said secondary antibody is labeled with a detectable label or is conjugated to an enzyme that generates a detectable compound from a precursor.

In a preferred embodiment, the solid support is a solid phase of an affinity column and said method further comprises a step of eluting the antibody or antibody fragment from the affinity column.

In a preferred embodiment, the similarity between the both epitopes from Clostridium difficile toxin A or Clostridium difficile toxin B allows the use of a single epitope for an affinity purification of specific neutralizing antibodies.

The antibody or antibody fragment obtained or obtainable from a method of the present invention may be monoclonal or polyclonal. This may also depend on the fluid used as source. In a preferred embodiment, the antibody or antibody fragment of the present invention is monoclonal. In another preferred embodiment, the antibody or antibody fragment of the present invention is polyclonal.

In a preferred embodiment, the method of the present invention further comprises a step of isolating or removing one or more antibody classes selected from the group consisting of IgG, IgM, IgD, IgE and IgA.

The immunoglobulin may be IgA, where IgA may be monomeric, dimeric, polymeric, contains an additional J-chain, recombinant, comprises a secretory component, is administered orally as tablet or capsule or via inhalation. The immunoglobulin may be IgM, where IgM may be monomeric, dimeric, polymeric, contains an additional J-chain, recombinant, comprises a secretory component, is administered orally as tablet or capsule or via inhalation. The immunoglobulin may be IgG, where IgG may be monomeric, dimeric, polymeric, contains an additional J-chain, recombinant, comprises a secretory component, is administered orally as tablet or capsule or via inhalation.

The immunoglobulin may be a hyper-immune antibody that may be directly isolated from plasma and/or all its derivatives, fractions and waste fractions during plasma fractionation.

In a preferred embodiment, the method of the present invention further comprises a step of detecting the bound antibody or antibody fragment.

In a preferred embodiment, the step of detecting the bound antibody or antibody fragment comprises the following steps.
(a) binding a secondary antibody selectively to the Fc part of the bound antibody or antibody fragment; and
(b) detecting said secondary antibody.

In a preferred embodiment, the secondary antibody is labeled with a detectable label or is conjugated to an enzyme that generates a detectable compound from a precursor.

For instance, the secondary antibody may be labelled by a fluorescent dye (including small-molecule dyes, quantum dots, fluorescent proteins, etc.), a metal bead (e.g., gold beads) by an enzyme that generates a detectable signal (e.g., horseradish peroxidase (HRP)).

A still further aspect of the present invention relates to a method for testing the ability of an antibody or antibody fragment for neutralizing the bioactivity of Clostridium difficile toxin A, Clostridium difficile toxin B or a combination of both, wherein said method comprises the following steps:
(A) providing:
   adherent mammalian cells in a cell culture,
   Clostridium difficile toxin A, Clostridium difficile toxin B or both, and the antibody or antibody fragment;
(B) contacting the Clostridium difficile toxin A, Clostridium difficile toxin B or combination of both and the antibody or antibody fragment with the adherent mammalian cells;
(C) incubating the exposed mammalian cells for a time sufficient for detachment of cells of lower viability; and
(D) detecting the degree of cell rounding,
   wherein the degree of cell rounding indicates the degree of remaining bioactivity of the Clostridium difficile toxin A, Clostridium difficile toxin B or both.

It will be understood that all definitions are preferred embodiments as indicated above mutatis mutandis also apply to the method. This method preferably is an in vitro method.

A still further aspect of the present invention relates to the antibody or antibody fragment of the present invention or an antibody obtained from a method of the present invention for use in a method for treating or preventing an individual suffering from a Clostridium difficile infection or being of risk of developing a Clostridium difficile infection.

It will be understood that all definitions are preferred embodiments as indicated above mutatis mutandis also apply to the antibody or antibody fragment for use.

In other words, the present invention also relates to a method for treating or preventing an individual suffering from a Clostridium difficile infection or being of risk of developing a Clostridium difficile infection in a patient, wherein said patient is administered with a sufficient amount of antibody or antibody fragment of the present invention or an antibody obtained from a method of the present invention.

The term “suffering from” as used herein may be understood in the broadest sense in a way that the patient has developed a pathological condition associated with Clostridium difficile. The patient suffering from a disorder not necessarily but optionally bears medicinal symptoms.

The term “being at risk of” or “being at risk of developing” means that the patient has a certain risk of having a disorder associated with Clostridium difficile.

The present invention also includes the neutralization and treatment of acute/chronic gastrointestinal infections/inflammations caused by bacteria, viruses and their toxins, like Clostridium difficile toxins A and B and all their subtypes by the following mechanics:

a) The blocking of receptor bindings sites, hindering the entrance of toxins into the cells of the gastrointestinal tract of the human body.
b) By sterically blocking the one or more of the four subdomains of the toxins, inhibiting their enzymatic activity. For example, by blocking the GTP- and ATP-domain of toxins A and B of Clostridium difficile from interacting with the intracellular Rho family proteins rendering the toxin containing endosome harmless and preventing cytopathic effects and apoptosis.

The present invention may also include the following indications:
Gastrointestinal diseases caused by bacteria or viruses, like CDAD, with a focus on severe cases where the treatment with antibiotics would cause a high risk for the patient due to the release of clostridial spores and toxins.
Furthermore, the treatment in cases of acquired antibiotics resistance, where the current clinical standard treatment shows no effect on the patient.
The screening of patients to generate separated pools of hyperimmunized donors against gastrointestinal diseases caused bacteria and viruses. The screenings may be performed by identifying the high-affinity and neutralizing epitope via techniques, like PepScan and others. The identified epitopes may be created artificially and can be bound onto a solid phase to bind immunoglobulins with the respective affinity including neutralizing properties. The antibodies without or with lower affinities may be washed away and remaining high-affinity (including neutralizing) antibodies may be detected using secondary antibodies with a linked reporter enzyme.
Testing of efficacy of hyperimmune for antibodies for Mode of Action-studies in animals (mice, rats, hamsters etc.) who are challenged with the toxins and spores of the respective bacteria or viruses.

A still further aspect of the present invention relates to the use of an antibody or antibody fragment of the present invention or an antibody obtained from a method of the present invention for detecting Clostridium difficile toxins A and B in a fluid. Such detection may be performed by any of the means laid out above.

The invention is further explained by the following examples, claims and Figures, which are intended to illustrate the present invention.

### BRIEF DESCRIPTION OF THE FIGURES

FIG. 1 and FIG. 2 show, visualized from different angles, the location of epitopes according to preferred embodiments (depicted in black) of the present invention at the outer surface of the glucosyltransferase-, cysteine-protease- and translocation-domain of Clostridium difficile toxin A (depicted in grey).

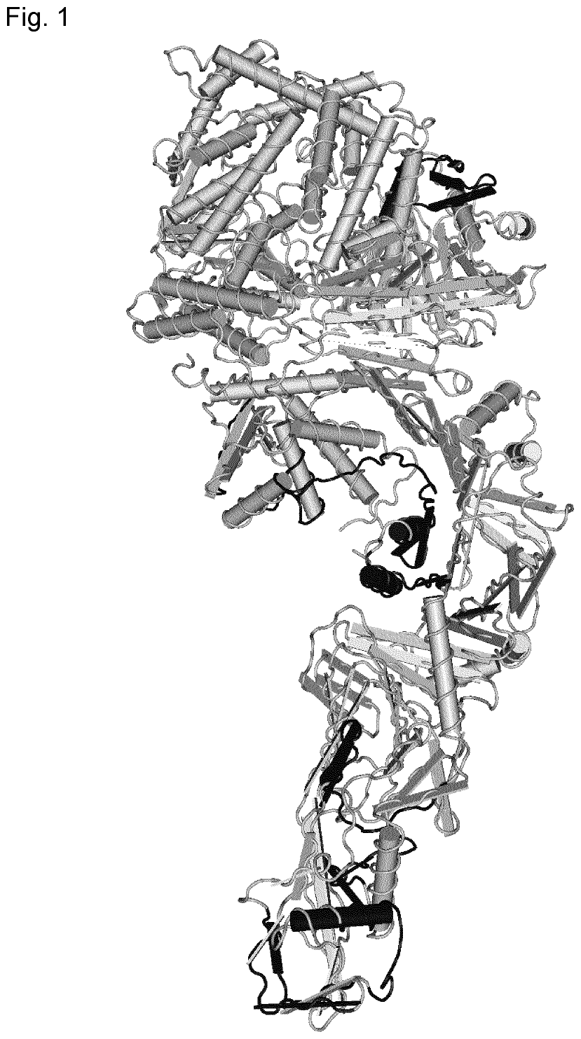

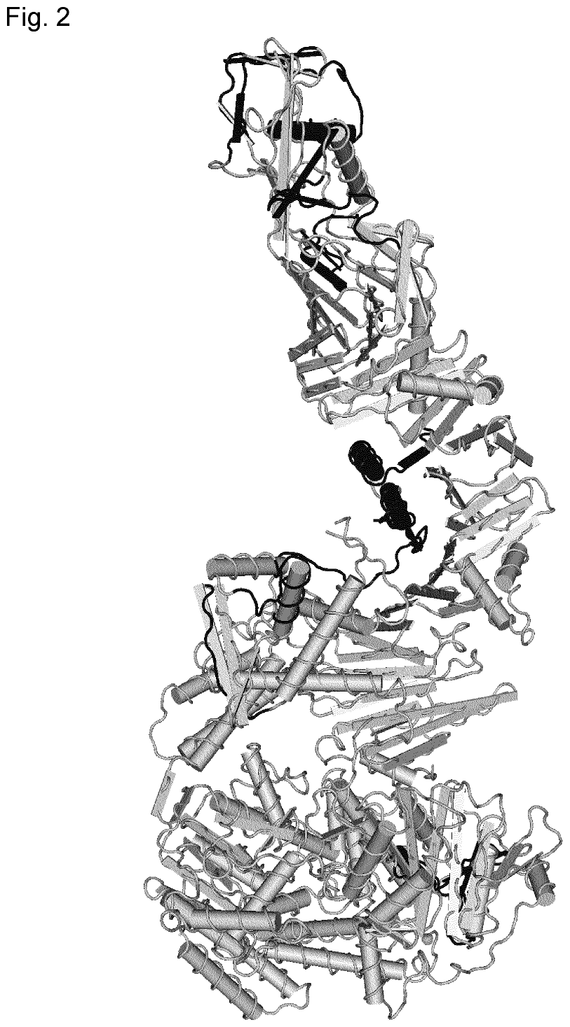

### EXAMPLES

Polypeptides as described above, for instance polypeptides of SEQ ID NO: 2 to 10, are synthesized by means of solid phase peptide synthesis (SPPS) or obtained from recombinant expression.

### Example I Positive Affinity Chromatography at an Epitope

A polypeptide as described above, for instance a polypeptide of SEQ ID NO: 2 to 10, is immobilized on an affinity chromatography matrix (affinity beads). This material is filled in an affinity chromatography column. The column is washed with a buffer (PBS). The column is contacted with the body fluid (blood serum or a fraction thereof).

The antibodies and antibody fragments specifically binding to the respective polypeptide comprising the epitope bind to their targets in the affinity column. The column is washed by a flow-through of buffer. Thereby unbound components including unbound antibodies and antibody fragments having no or merely a low affinity to the polypeptide are removed.

Subsequently, the specific antibodies and antibody fragments are eluted either by acetic buffers (e.g. 0.1 mol/L glycine/HCl pH 2.0) or by 3 mol/L KSCN, or by 8 mol/L urea. Thereby, the specifically binding antibodies are isolated.

### Example II IgA Separation

IgA is isolated direct from plasma of from plasma fractions which are purified from IgG. When those fractions are used, the separation of the specific immunoglobulin classes is not needed. When the positive affinity chromatography is performed ahead of the separation of the immunoglobulin classes, the separation of them is performed in a second step.

An example for the separation of (hyperimmune) IgA includes subjecting a plasma pool to Cohn fractionation. This is followed by an IgG polishing step providing IgG and an IgA/IgM fraction. The IgA/IgM fraction is subjected to affinity chromatography as described above and provides (hyperimmune) IgA.

An alternative example for the separation of (hyperimmune) IgA includes subjecting an unfractioned plasma pool subjected to affinity chromatography as described above. This provides an immunoglobulin fraction (including IgG and IgA) and a residual blood fraction which is further subjected to Cohn fractionation. The immunoglobulin fraction (including IgG and IgA) is subjected to a second affinity chromatography separating IgG and IGA and provides (hyperimmune) IgA.

### Example III Separation of Plasma Containing (Neutralizing) Antibodies Against Clostridium difficile Toxin a and/or Clostridium difficile Toxin B

The donations (plasma and or blood) are screened by commercially available ELISA. Here, the Clostridium difficile toxin A or B is insolubilized at a solid phase (e.g. microtiter plate) (Porstmann et al., “Enzyme immunoassay techniques. An overview”. Journal of Immunological Methods, 1992, 150:5-21). The specific antibodies of different immunoglobulin classes are separated by using different class-specific antibodies in a labelled form (e.g. anti-IgG-HRP, anti-IgA-HRP).

Due to the fact, that the immune reaction shows stable antibody responses even month and years after an infection, a quarterly or half-year screening of donors can be performed. The selected donations are pooled and used in the plasma fractionation for the separation of all other plasma proteins. The IgA containing fraction is used separately.

### Example IV Enzyme-Linked Immunosorbent Assay (ELISA)

A polypeptide as described above, for instance a polypeptide of SEQ ID NO: 2 to 10, is immobilized on a bottom of a microtiter plate. The body fluid (blood fraction, blood plasma/serum) is contacted for several minutes. Then, the microtiter plate is washed with buffer (PBS) and contacted with an enzyme-labelled secondary antibody (e.g., conjugated with HRP). A substrate suitable to be converted into a detectable moiety by the enzyme is added and the staining of the microtiter plate is performed in a plate reader. This assay provides an antibody titer of the body fluid.

### Example V Cell-Based Neutralization Assay

The ability of antibodies to neutralize the toxins is tested via the exposal of mammalian cells to one of the toxins (HT29 for Clostridium difficile toxin A and CHO for Clostridium difficile toxin B). The CHO cells are grown in DMEM/Ham's F12 and HT29 in McCoys 5A in in cell culture dishes and supplemented 20% fetal calf serum and glutamine. The toxins and the mono- or polyclonal antibodies or antiserum are incubated for 60 min at 37° C. On the first day, the cells are seeded in a 96 well cell culture plates and incubated overnight. Dilutions of antibodies and LCTs are done in the cell culture medium. The toxins concentrations for the assay are chosen in a way that just enough toxin is administered to induce complete rounding overnight. On the second day, the diluted antibodies are mixed with subsequent dilutions of the LCTs. After incubation (60 min at 37° C.) of the antibody-toxin mixes are added to the cells and cell rounding is observed after 20-24 hours. As control, toxin is added to the cells without antibodies.

The rounding is evaluated by microscopic analysis on the third day, using the following criteria:

(−) no cell rounding
(+) ≤10% cell rounding
(++) >10% cell rounding
(+++) 85-100% cell rounding.

### Example VI Animal (Hamster) Model

The primary objective of such a study is to evaluate the dose of specific human IgA antibodies against Clostridium difficile toxin A and/or B in an oral therapy. In a three-armed feasibility animal study specific IgA against the Clostridium difficile toxins and standard therapy are compared. Any prolongation of life span is the primary measure the secondary is survival at day 24.

### Study Design and Methodology:

IgA is prepared using common methods from plasma of normal healthy plasma donors. The donors have previously been screened first for the presence of specific antibodies against Clostridium difficile toxin A and B and antibodies against Clostridium difficile. IgA is enriched from those donations. Such IgA-enriched plasma-fraction is stored frozen until use in single does, each at 250 μL with 1 mg/mL. The IgAs is administered into Hamster free of Clostridium difficile (Checked by NAT at d−4 . . . d−6, at tgcBIOMICS GmbH)

### Eight Male Animals are Used Per Group:

(A) Disease control group: remains completely untreated after Clindamycin spores application—time to death of hamsters is determined in this untreated control group. (B) Vancomycin-treatment group: 3-days of treatment with Vancomycin (10 mg/kg administered p.o. starting at day d+2), prolongation of life span is monitored. (C) Plasma-treatment group: treatment with Vancomycin (10 mg/kg, at days +2 till +4) is performed like for group (B), but is accompanied by treatment at days +3 till +6 with specific IgA-enriched plasma-concentrate which contains neutralizing antibodies against Clostridium difficile toxin A and B

Animals are housed in socially harmonious groups of four animals in individual ventilated cages with a surface area of 1500 cm². Housing in groups becomes appropriate, if no animal is positive in the NAT for of C. diff genes. The general colonisation status of the animals is checked with the 24 animals in the forefront of starting the animal experiment.

To change the intestinal flora, the animal is treated at d0 with Clindamycin i.p. One day later (d0) the animal is challenged with 100 to 1000 spores of C. diff (strain: 630). Groups (B) and (C) receive a 3-day vancomycin treatment which reduces the bacterial load following infection. The IgA-enriched Plasma-treatment starts at d2 with 2 doses IgA for four days in total (d2: 2 doses enriched-IgA; d3: 2 doses enriched-IgA, d3: 2 doses enriched-IgA, d4: 2 dose2 enriched-IgA).

The animals are regularly observed for body temperature by infrared temperature measurement, stool consistency, clinical signs, and every 2^{nd} day for their body weight. Stool samples are taken ahead of the experiment at d−4 . . . d−6 for C. diff-NAT-testing and at d+3; d+6 and d+10 to test for the presence of human IgA in stool and the detection of blood in the stool. Blood samples are taken at d−6 . . . d+7, d+14 . . . and at d+21 from orbital plexus for the detection of any specific antibodies induced in hamsters against both toxins and Clostridium difficile. The animals are euthanized when the weight loss exceeds 60%. The total observation time is 24 days. After necropsy, signs of inflammation and the length of the intestine have to be documented. Stool is collected of the individual animals. The animal receiving specific IgA against Clostridium difficile toxins A and B are protected and survive the observation period.

1-16. (canceled)

17. A polypeptide comprising at least one epitope, wherein the epitope is at least eight consecutive amino acids in length and the epitope has a sequence homology of at least 75% to a sequence section of Clostridium difficile toxin A and a sequence homology of at least 75% to a sequence section of Clostridium difficile toxin B, and wherein the polypeptide has a length of 8 to 100 consecutive amino acid moieties, or an immunogenic peptidomimetic or retro-inverso polypeptide thereof.

18. The polypeptide of claim 17, wherein the polypeptide is obtained by means selected from the group consisting of polypeptide synthesis, gene technological means, and isolation of Clostridium difficile toxin A or Clostridium difficile toxin B and subsequent protein digestion.

19. The polypeptide of claim 17, wherein the epitope has a sequence homology of at least 75% to a sequence section located at the outer surface of Clostridium difficile toxin A and has a sequence homology of at least 75% to a sequence section located at the outer surface of Clostridium difficile toxin B.

20. The polypeptide of claim 17, wherein the epitope has or comprises an amino acid sequence selected from the group consisting of
(SEQ ID NO: 3)
ANQYEVRINSEGRX²³ELLX¹HSGX²⁵WINKEEX²⁶IX²¹;
(SEQ ID NO: 4)
GESX²¹X²⁷VETEK;
(SEQ ID NO: 5)
X²⁶X²¹KVQX²¹YAQLFSTGLNTI;
(SEQ ID NO: 6)
LX²¹PX²¹AGISAGIPSLVNNELX²¹L;
(SEQ ID NO: 7)
DDLVISEIDFNNNSI;
(SEQ ID NO: 8)
MEGGSGHTVTX¹X²⁵IDHFFSX²⁶PSIX²²;
(SEQ ID NO: 9)
PGLRSLENDGTX²³LLD;
and
(SEQ ID NO: 10)
AX²¹X²⁵X²⁴TIX²⁵X²¹LPTX²¹X²²EGX²¹PIX²¹X²⁶TIX²¹DGX²¹
X²²LGAAIKELX¹X²⁴X¹X¹DPLLX²³X²⁵EX²¹EAKX²¹GX²¹X²¹A
X²¹NX²¹X²²,
wherein:
X¹ is any naturally occurring amino acid moiety or is a direct bond between the adjacent amino acid moieties;
X²¹ is an amino acid moiety selected from the group consisting of G, A, V, P, L, I, M, W, and F;
X²²: is an amino acid moiety selected from the group consisting of S, T, Y, C, N, U, O, and Q;
X²³ is an amino acid moiety selected from the group consisting of K, R, and H;
X²⁴ is an acidic amino acid moiety selected from the group consisting of D and E;
X²⁵ is an amino acid moiety selected from the group consisting of S, T, Y, C, N, Q, K, R, H, U, O, D, and E;
X²⁶ is an amino acid moiety selected from the group consisting A, G, and S; and
X²⁷ is an amino acid moiety selected from the group consisting of Y, G, A, V, P, L, I, M, W, and F,
or in an immunogenic peptidomimetic or retro-inverso polypeptide thereof.

21. The polypeptide of claim 17, wherein the polypeptide is immobilized on a solid support.

22. A vaccine comprising at least one polypeptide of claim 17 and at least one pharmaceutically acceptable carrier.

23. A method for preventing an individual from developing a Clostridium difficile infection, comprising administering the individual with a sufficient amount of the vaccine of claim 22.

24. An antibody or antibody fragment binding to Clostridium difficile toxin A with a dissociation constant Kd of less than 100 nM and to Clostridium difficile toxin B with a dissociation constant Kd of less than 100 nM.

25. A method for isolating and/or detecting an antibody or antibody fragment binding to Clostridium difficile toxins A and B from a fluid containing the antibody or antibody fragment, wherein the method comprises the following steps:
(i) providing:
the fluid containing the antibody or antibody fragment, and
a polypeptide according to claim 17 immobilized on a solid support;
(ii) contacting the fluid with the immobilized polypeptide and allowing the antibody or antibody fragment to bind to the immobilized polypeptide; and
(iii) removing at least parts of the unbound fluid and optionally washing the solid support with a fluid not containing the containing the antibody or antibody fragment.

26. The method of claim 25, wherein the solid support is a solid phase of an affinity column.

27. The method of claim 25, wherein the fluid is a body fluid.

28. The method of claim 25, wherein the method further comprises a step of isolating or removing one or more antibody classes selected from the group consisting of IgG, IgM, IgD, IgE, and IgA.

29. The method of claim 25, wherein the method further comprises a step of detecting the bound antibody or antibody fragment.

30. A method for testing the ability of an antibody or antibody fragment for neutralizing the bioactivity of Clostridium difficile toxin A, Clostridium difficile toxin B or a combination of both, wherein the method comprises the following steps:
(A) providing:
adherent mammalian cells in a cell culture,
Clostridium difficile toxin A, Clostridium difficile toxin B or both, and
the antibody or antibody fragment;
(B) contacting the Clostridium difficile toxin A, Clostridium difficile toxin B or combination of both and the antibody or antibody fragment with the adherent mammalian cells;
(C) incubating the exposed mammalian cells for a time sufficient for detachment of cells of lower viability; and
(D) detecting the degree of cell rounding,
wherein the degree of cell rounding indicates the degree of remaining bioactivity of the Clostridium difficile toxin A, Clostridium difficile toxin B, or both.

31. A method for treating or preventing an individual suffering from a Clostridium difficile infection or being of risk of developing a Clostridium difficile infection, comprising administering the individual with a sufficient amount of the antibody or antibody fragment of claim 24.

32. A method for treating or preventing an individual suffering from a Clostridium difficile infection or being of risk of developing a Clostridium difficile infection, comprising administering the individual with a sufficient amount of an antibody or antibody fragment obtained from a method of claim 25.

33. The antibody or antibody fragment of claim 24, wherein the antibody or antibody fragment binds to an epitope with a dissociation constant Kd of less than 100 nM,
wherein the epitope has a sequence homology of at least 75% to a sequence section of Clostridium difficile toxin A and a sequence homology of at least 75% to a sequence section of Clostridium difficile toxin B, and wherein the polypeptide has a length of 8 to 100 consecutive amino acid moieties, or an immunogenic peptidomimetic or retro-inverso polypeptide thereof.

34. The method of claim 25, wherein the antibody or antibody fragment has a dissociation constant Kd of less than 100 nM and to Clostridium difficile toxin B with a dissociation constant Kd of less than 100 nM.

35. The method of claim 26, wherein the method further comprises a step of eluting the antibody or antibody fragment from the affinity column.

36. The method of claim 27, wherein the fluid is a body fluid selected from the group consisting of blood plasma and a fraction of blood plasma.

37. The method of claim 27, wherein the method further comprises preparing of a fraction of blood plasma by a Cohn or Kistler-Nitschmann process.

38. The method of claim 29, wherein the step of detecting the bound antibody or antibody fragment comprises the following steps:
(a) binding a secondary antibody selectively to the Fc part of the bound antibody or antibody fragment; and
(b) detecting the secondary antibody.

39. The method of claim 38, wherein the secondary antibody is labeled with a detectable label or is conjugated to an enzyme that generates a detectable compound from a precursor.
